(19) United States
(12) Patent Application Publication
(10) Pub. No.: US 2011/0091518 A1
(43) Pub. Date: Apr. 21, 2011

(54) IMPLANT DEVICES HAVING VARYING BIOACTIVE AGENT LOADING CONFIGURATIONS

(76) Inventor: Danielle Biggs, Collierville, TN (US)

(21) Appl. No.: 12/887,893

(22) Filed: Sep. 22, 2010

Related U.S. Application Data
(60) Provisional application No. 61/244,736, filed on Sep. 22, 2009.

(51) Int. Cl. A61F 2/00

(52) U.S. Cl. 424/426

(57) ABSTRACT

Described herein are implant devices comprising various configurations of bioactive agent loading which can be selected and used to tailor a particular bioactive agent release profile from the implant device.

### CROSS REFERENCE TO RELATED APPLICATIONS

This application is based upon and claims the benefit of priority from prior U.S. Provisional Application No. 61/244,736, filed Sep. 22, 2009, the entire contents of which are incorporated herein by reference.

### BACKGROUND

In the realm of pharmaceutical formulations, there is a class of drug-delivery formulations that are designed to release bioactive agents for a desired period of time following a single administration. Depot formulation is one name used to describe these long-acting formulations. Depot formulations can be fabricated in many ways. A typical formulation approach to prepare a depot formulation or implant is by manufacturing a solid matrix that includes a bioactive agent and a polymeric excipient. The purpose of the polymeric excipient of the implant is to restrict the influx of water, which in turns controls the dissolution of the bioactive agent followed by the release of the bioactive agent from the implant matrix. In addition to the physical and chemical properties of the bioactive agent, the amount of bioactive agent in the implant contributes to the rate of bioactive agent release. That is, increasing the amount of bioactive agent increases the rate of release. Unfortunately, some implant formulations require a high amount of bioactive agent inside in order to have enough bioactive agent available to achieve dose and duration requirements for a particular medical indication. A high amount of bioactive agent incorporated inside the implant, however, may cause the release the bioactive agent to occur too fast or even at an uncontrollable rate.

As such, there is a need for new implant devices which can be loaded with varying, including high, amounts of bioactive agent yet still maintain a satisfactory release, such as an extended release profile or a release profile with a low initial burst, among others. These needs and other needs are satisfied by the present invention.

### SUMMARY

Described herein are implant devices comprising various configurations of bioactive agent loading which can be selected and used to tailor a particular bioactive agent release profile from the implant device.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 is an isometric cross-sectional view of an exemplary implant device having a core surrounded by a membrane shell.

FIG. 2 is a top cross-sectional view of a coextrusion apparatus that can be used to make implant device having a core surrounded by a membrane shell.

### DETAILED DESCRIPTION

Before the present compounds, compositions, composites, articles, devices and/or methods are disclosed and described, it is to be understood that the aspects described below are not limited to specific compounds, compositions, composites, articles, devices, methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

A “releasable agent” refers to an agent that can be mixed together with a disclosed polymer and subsequently released therefrom, for example, as the polymer erodes.

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. A “releasable bioactive agent” is one that can be released from a disclosed polymer. Bioactive agents also include those substances which affect the structure or function of a subject, or a pro-drug, which becomes bioactive or more bioactive after it has been placed in a predetermined physiological environment.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Generally, the implant devices of the invention comprise a longitudinal body and proximal and distal ends (and proximal and distal end surfaces). The longitudinal body comprises a biocompatible and/or biodegradable polymer. The longitudinal body comprises a longitudinal core surface, which can be (i) a partially or completely exposed surface, (ii) partially or completely coated with a bioactive agent, (ii) partially or completely surrounded (i.e., not exposed) by a polymeric sheath (which can contain or be free of bioactive agent and the surface of which can be coated or can be free of bioactive agent) or a combination of (i), (ii), and (iii).

The implant device is loaded with a bioactive agent according to a particular loading configuration depending on the desired release profile. By varying the bioactive agent loading configuration in the implant devices of the invention, release profiles can be tailored to a specific need, and sophisticated release profiles can be achieved.

Generally, the bioactive agent can be present in (i.e. within the longitudinal body and/or polymeric sheath) or on any surface of the implant. The bioactive agent can generally be (i) coated onto only one or more of the proximal or distal end surfaces, (ii) coated onto one or more of the proximal or distal end surfaces and only a portion, or all, of the outer surface of the longitudinal body, (iii) coated onto a portion or all of the longitudinal body but not coated onto either end surface, (iv) dissolved or dispersed in the inner core (when present), (v) dissolved or dispersed in the longitudinal body, (vi) dissolved or dispersed in the polymeric sheath (when present), (vii) absent from the polymeric sheath (when present), or any combination of (i)-(viii).

In one aspect, the implant device can be bulk-loaded. In this aspect, the bioactive agent is dissolved or dispersed throughout the longitudinal body. The surfaces of the implant device can be coated with bioactive agent, or can be free of bioactive agent. This aspect can include examples wherein the longitudinal body forms an inner core and is surrounded by a polymeric sheath.

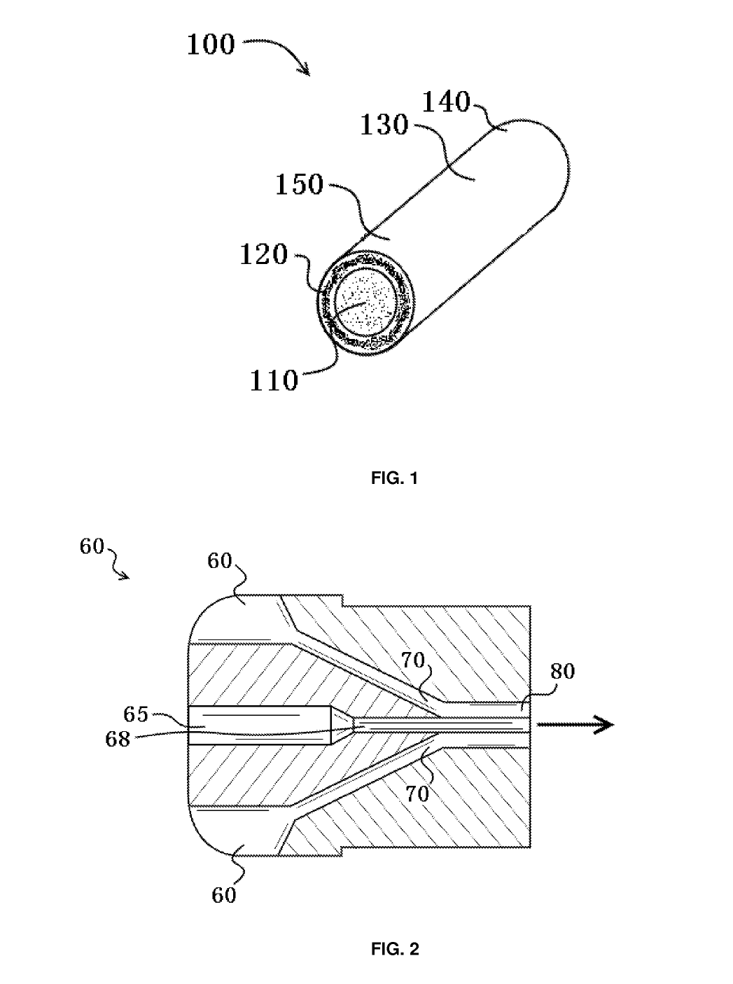

In another aspect, the longitudinal body comprises an inner core having a longitudinal core surface surrounded by a polymeric sheath and has exposed proximal and distal end surfaces that are not surrounded by the polymeric sheath. The polymeric sheath comprises a longitudinal outer surface which is substantially coextensive with the longitudinal core surface. The inner core comprises a biodegradable polymer having a bioactive agent dissolved or dispersed therein. In one example, the polymeric sheath is free of bioactive agent. In other examples, the polymer can contain bioactive agent dissolved or dispersed therein. With reference to FIG. 1, for example, the implant device 100 comprises a longitudinal body 130 comprising an inner core 110 which is loaded with bioactive agent, and a longitudinal core surface which is surrounded and coextensive with a polymeric sheath 150, which comprises an outer polymeric sheath surface 140. The implant device also comprises a coating 120 of bioactive agent on the proximal and/or distal end surfaces, including the portion of the end surface formed by the outer polymeric sheath (but not within the polymeric sheath) and the portion of the end surface formed by the inner core. In a similar embodiment, the bioactive agent can also be coated onto the longitudinal surface in addition to being coated onto the end surface. In another embodiment, the bioactive agent can be coated onto the longitudinal surface and not coated onto the proximal and distal surfaces. In still another embodiment, the bioactive agent can be present within (i.e., dissolved or dispersed) both the core and the polymeric sheath. In this embodiment, the concentration of the drug in the core and the surrounding polymeric sheath can be the same or different.

In another aspect, the longitudinal body comprises an inner core having a longitudinal core surface surrounded by a polymeric sheath and has exposed proximal and distal end surfaces that are not surrounded by the polymeric sheath. The polymeric sheath comprises a longitudinal outer surface which is substantially coextensive with the longitudinal core surface. The inner core comprises a biodegradable polymer and is free of bioactive agent, or does not have bioactive agent dissolved or dispersed therein. In this aspect, the bioactive agent can be coated onto one or more of the outer surfaces, including one or more of the longitudinal outer surface, the proximal end surface, the distal end surface, or a combination thereof, including those examples wherein the bioactive agent is coated onto a part or all of every exposed surface of the implant device.

In another aspect, the implant device comprises a longitudinal body which can have a longitudinal surface that is or is not surrounded by a polymeric membrane sheath and thus is exposed. In this aspect, the longitudinal body dissolved or dispersed therein, and bioactive agent is present only on one or more of the proximal or distal end surfaces.

An implant device having a core/sheath arrangement, in one aspect, can be prepared by a process comprising: a. forming a core having a desired shape from an admixture of a biodegradable polymer and optionally bioactive agent (if inner core loading is desired); b. forming a membrane sheath surrounding the core; and c. exposing the proximal and distal end surfaces by removing that portion of the membrane sheath that surrounds the end surfaces.

For a core/sheath configuration wherein bioactive agent is dissolved or dispersed in the inner core, forming the core of the implant device can be accomplished by first admixing at least one biodegradable polymer and at least one bioactive agent to produce an admixture. The admixing of the biodegradable polymer and the bioactive agent can be performed using techniques known in the art. For example, the polymer and agent can be dry blended (i.e., mixing of particulates of the polymer and the agent) using, for example, a Patterson-Kelley V-blender, or granulated prior to processing step prior to forming the desired-shaped core. It is contemplated that other components such as, for example, excipients, can be admixed with the polymer and the agent prior to processing the admixture into a core.

The admixing step can include the use of a solvent. In other aspects, however, the admixing of the biodegradable polymer and the bioactive agent does not involve the use of a solvent. A number of advantages can be realized when avoiding the use of a solvent during admixing. First, the use of a solvent during admixing requires additional processing steps to remove the solvent. Second, if the delivery system is to be implanted into a subject, the selected solvent has to be biocompatible if any residual solvent remains in the device. The solvent can adversely affect the overall morphology of the delivery system, which can lead to undesirable release patterns. The solvent can adversely affect the stability of the bioactive agent during the manufacturing process. Finally, the solvent level requires control, because it has to be low enough to meet regulatory guidelines.

The processing of the admixture into the inner core can can be performed under conditions such that the bioactive agent is intimately mixed, dispersed, or dissolved throughout the polymer or in only certain portions of the polymer. The admixture can be processed into the desired shaped inner core by a variety of techniques, such as, for example, melt extruding, injection molding, compression molding, or roller compacting the admixture into a desired shape or structure. Compression manufacturing techniques can include, but are not limited to tabletting. Depending upon processing conditions, the biodegradable polymer used as a starting material in the admixing step may or may not be the same polymer present in the final device. For example, the polymer during processing may undergo polymerization or depolymerization reactions, which ultimately can produce a different polymer that was used prior to processing. Thus, the term “polymer,” including both the biocompatible polymer and the biodegradable polymer, as used herein covers the polymers used as starting materials as well as the final polymer present in the final device.

In one aspect, the inner core having a desired shape is first processed as discussed above (with or without the bioactive agent), and then the membrane sheath that surrounds core is formed. In other aspects discussed below, the inner core and membrane sheath can be coprocessed, for example, through coextrusion to provide the implant device. When the inner core is first formed, the membrane sheath can subsequently be formed using methods known in the art. In one aspect, the membrane sheath can be formed by spray-coating or dip-coating a solution comprising the biocompatible polymer (and optionally a bioactive agent) onto the inner core. In this aspect, the membrane sheath can be formed around the entire inner core, such that the inner core does not have an exposed surface. After forming the membrane sheath, a portion of the membrane sheath can be removed, for example by dissolving away or physically cutting away a portion of the membrane sheath to provide an exposed inner core surface (i.e., the proximal or distal end surface). In other aspects, a membrane sheath can be formed surrounding only a portion of the core such that the core comprises an exposed surface after forming the membrane sheath.

In another aspect, the implant device can be prepared by coextrusion, for example by a process comprising: a. extruding a biodegradable polymer, or in the alternative, an admixture of a biodegradable polymer and a bioactive agent, through an inner coaxial nozzle to form a core; b. forming a composite strand by simultaneously coextruding a biocompatible polymer, or in the alternative, an admixture of a biocompatible polymer and a bioactive agent, through an outer coaxial nozzle to apply a substantially coextensive membrane sheath surrounding the core; c. cutting the composite strand of step and (b) into one or more slats comprising a longitudinal surface and two end surfaces. An implant device as shown in FIG. 1, for example, can be prepared by this method.

With reference to FIG. 2, the coextrusion method can be accomplished with a variety of coextrusion devices known in the art. FIG. 2 shows a cross-section 60 of such a device. In the coextrusion process, the polymer or admixture, which can be formed as discussed above, is flowed through an inner coaxial nozzle 65, while the biocompatible polymer or admixture that will form the membrane sheath is flowed through an outer coaxial nozzle 60. The inner 65 and outer 60 coaxial nozzles can then narrow into mold sections 68 and 70, where the biocompatible polymer or admixture and the biodegradable polymer or biodegradable polymer/bioactive agent admixture are combined and shaped into the desired shape of the implant device, which in this example is a cylinder. The coextruded composite strand then exists the device at exit point 80. After coextrusion, the coextruded composite strand can be cut into one or more slats comprising a longitudinal surface and two end surfaces, as discussed above and as shown in FIG. 1. Thus, after cutting the coextruded strand, the implant device can be formed by cutting the strand into individual slats, which each comprise a longitudinal surface and a proximal and distal end surface, as discussed above. The strand can be cut into as many slats as desired, to produce a desired number of implant devices, or implant devices of a desired longitudinal length.

The implant devices that are not of core/sheath arrangement can be prepared by more simplified extrusion methods, for example using single-mold extrusion, and cut into one or more slats as discussed above.

The implant devices, in some aspects, comprise coatings of the bioactive agent on or more surfaces of the device. The bioactive agent coating can be applied to the implant device by preparing an appropriate solution of dispersion of the bioactive agent in a solvent and subsequently applying the solution to the one or more exposed surfaces of the implant device. The application of the solution can be carried out by spraying, dipping, brushing, etc., the solution onto the desired surface of the implant device, following by allowing the solvent to evaporate, if desired.

A variety of biocompatible or biodegradable polymers can be used to form the implant devices, including those used for the membrane sheath and/or used as the polymer of the inner core. The biocompatible polymer can also be a biodegradable polymer. In one aspect, the biocompatible polymer can be one or more of polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable and bioacompatible polymers include without limitation ethylene-vinyl acetate co-polymer, polytetrafluoroethylene, polypropylene, polyethylene, and the like. Likewise, other suitable non-biodegradable polymers include without limitation silicones and polyurethanes.

The biodegradable polymer that forms the inner core or membrane sheath (when present) can include any of those biodegrable polymers listed above or any other biodegradable polymer known in the art. In a further aspect, the biocompatible and/or biodegradable polymer can be a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

In a still further aspect, useful biodegradable and biocompatible polymers are those that comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. In a still further aspect, useful biodegradable polymers are those that comprise one or more residues of lactide, glycolide, caprolactone, or a combination thereof.

In one aspect, useful biodegradable and biocompatible polymers are those that comprise one or more blocks of hydrophilic or water soluble polymers, including, but not limited to, polyethylene glycol, (PEG), or polyvinyl pyrrolidone (PVP), in combination with one or more blocks another biocompatible or biodegradable polymer that comprises lactide, glycolide, caprolactone, or a combination thereof.

In specific aspects, the biodegradable and/or biocompatible polymer can comprise one or more lactide residues. To that end, the polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

When the biodegradable and/or biocompatible polymer is poly(lactide-co-glycolide), poly(lactide), or poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

In a further aspect, the biodegradable and/or biocompatible polymer can be a poly(caprolactone) or a poly(lactide-co-caprolactone). In one aspect, the polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

When either the biodegradable or biocompatible polymers comprise lactide-based polymers, the lactide-based polymers can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be made by ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

In some aspects, it can be desirable to contact or admix a disclosed biodegradable and/or biocompatible polymer with one or more plasticizers, in order to alter the physical properties (e.g., lower the T_{g}) of the resulting composition. Plasticizers that can be used include all FDA approved plasticizers, such as benzyl benzoates, cellulose acetates, cellulose acetate phthalates, chlorobutanol, dextrines, dibutyl sebacate, dimethyl sebacate, acetyl phthalates, diethyl phthalate dibutyl phthalate, dipropyl phthalate, dimethyl phthalate, dioctyl phthalate, methyl cellulose, ethyl cellulose, hydroxylethyl cellulose, hydroxypropyl cellulose, hydroxypropyl methyl celluloses, gelatine, glycerines, glyceryl monostearate, monoglycerides, mono and di-acetylated monoglycerides, glycerol, mannitol, mineral oils and lanolin alcohols, petrolatum and lanolin alcohols, castor oil, vegetable oils, coconut oil, polyethylene glycol, polymethacrylates and copolymers thereof, polyvinyl-pyrrolidone, propylene carbonates, propylene glycol, sorbitol, suppository bases, diacetine, triacetin, triethanolamine, esters of citric acid, triethyl citrate, acetyl triethyl citrate, acetyl tributyl citrate, triethyl citrate, and esters of phosphoric acid.

The biodegradable polymer can erode and thereby allow the agent in the inner core of the implant device to be released. A variety of releasable agents can be used in the compositions. Generally, any agent for which release over time is desired can be used. Thus, the releasable agent can be a bioactive agent, cosmetic substance, such as a lotion, or other substance, such as an agricultural product. The releasable agent can be dissolved or dispersed in the polymer and can be present in any suitable amount, which will generally depend on the intended use of the composition.

A large variety of bioactive agents can be used with the implant devices. The bioactive agent can be blended, admixed, or otherwise combined with the biodegradable polymer of the inner core, membrane sheath, and/or be coated onto one or more surfaces, as discussed above. In one aspect, the bioactive agent can be preformulated, e.g., spray-dried with sugar, into a defined particle. In another aspect, at least a portion of the bioactive agent can be dissolved in the biodegradable polymer. In a further aspect, at least a portion of the bioactive agent can be dispersed in the biodegradable polymer of the inner core and/or membrane sheath (when present).

The admixing of the bioactive agent and the polymer can be carried out with or without an additional solvent (other than the polymer), as discussed above. The amount of bioactive agent incorporated into the composition varies depending upon a particular drug, the desired therapeutic affect and the desired time span. Because a variety of compositions are intended to provide dosage regimens for therapy for a variety purposes, there is no critical lower or upper limit in the amount of drug incorporated into the composition. The lower limit will generally depend upon the activity of the drug and the time span of its release from the device. Those skilled in the pharmaceutical arts can determine toxic levels of a given drug as well as the minimum effective dose.

Various forms of the bioactive agent can be used, which are capable of being released from the implant device into a subject. A liquid or solid bioactive agent can be incorporated into the devices described herein. The bioactive agents can be water soluble or water-insoluble. In some aspects, the bioactive agent is at least very slightly water soluble, and preferably moderately water soluble. The bioactive agents can include salts of the active ingredient. As such, the bioactive agents can be acidic, basic, or amphoteric salts. They can be nonionic molecules, polar molecules, or molecular complexes capable of hydrogen bonding. The bioactive agent can be included in the devices in the form of, for example, an uncharged molecule, a molecular complex, a salt, an ether, an ester, an amide, polymer drug conjugate, or other form to provide the effective biological or physiological activity.

Examples of bioactive agents that can be incorporated into the devices include, but are not limited to, small molecules, peptides, proteins such as hormones, enzymes, antibodies, antibody fragments, antibody conjugates, nucleic acids such as aptamers, iRNA, siRNA, DNA, RNA, antisense nucleic acid or the like, antisense nucleic acid analogs or the like, VEGF inhibitors, macrocyclic lactones, dopamine agonists, dopamine antagonists, low-molecular weight compounds, high-molecular-weight compounds, or conjugated bioactive agents. Bioactive agents contemplated for use in the disclosed compositions include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Other bioactive agents include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents in the compositions include, but are not limited to, peptide drugs, protein drugs, therapeutic antibodies, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, —adrenergic blocking agents, nutritional agents, and the benzophenanthridine alkaloids. The agent can further be a substance capable of acting as a stimulant, sedative, hypnotic, analgesic, anticonvulsant, and the like.

Other bioactive agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B₁₂, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxan®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the bioactive agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Tinidazole, Ropinerole, Ivermectin, Moxidectin, Afamelanotide, Cilengitide, or a combination thereof. In one aspect, the bioactive agent can be a combination of Rifampicin (Rifampin in U.S.) and Minocycline.

In some aspects, the device itself can be the carrier and/or can be combined with other carriers or additives. Other pharmaceutical carriers can also be used. Examples of solid carriers, other than the polymer (if solid), include lactose, terra alba, sucrose, talc, gelatin, agar, pectin, acacia, magnesium stearate, and stearic acid. Examples of liquid carriers, other than the polymer (if liquid), are sugar syrup, peanut oil, olive oil, and water. Examples of gaseous carriers include carbon dioxide and nitrogen. Other pharmaceutically acceptable carriers or components that can be mixed with the bioactive agent can include, for example, a fatty acid, a sugar, or a salt.

In one aspect, the composition can be present in a kit. The kit can comprise a suitable package or container for the compositions. Examples include without limitation sterile packaging. Because the disclosed compositions are suitable for use as injectable compositions, a kit can include a prepackaged injection device, comprising an injection device that is loaded with the implant device. Suitable injection devices include without limitation syringes, trochars, and others.

As discussed above, the implant devices can be used to administer a bioactive agent to a subject in need thereof, for example to treat a disorder for which the bioactive agent can effective. The compositions can be administered to any tissue or fluid of a subject. Likewise, the mode of administration can be any suitable mode, for example subcutaneous injection, oral administration, parental administration, enternal administration, and the like. In some aspects, the liquid compositions comprising one or more low viscosity polymers can be injected into a subject. The nature of the composition administered will generally be selected based on the desired dosage of the bioactive agent, which will vary greatly depending on the disorder but can be readily determined by one in the pharmaceutical arts.

An “effective amount” of a composition refers to an amount of the composition that will achieve a desired therapeutic result. Thus, the effective amount will vary greatly depending on the composition, bioactive agent, and disorder or condition that is being treated. The actual effective amount of dosage amount of the composition administered to a subject can be determined by physical and physiological factors such as body weight, severity of condition, the type of disease being treated, previous or concurrent therapeutic interventions, idiopathy of the patient and can depend on the route of administration. Depending upon the dosage and the route of administration, the number of administrations of a preferred dosage and/or an effective amount may vary according to the response of the subject. One of skill in the art can determine an effective amount of a disclosed pharmaceutical composition.

In some non-limiting examples, a dose can comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The bioactive agent can be present in the implant device in any suitable weight percent, including higher loading weight percents, such as up to 40% loading by weight of the implant device or by weight of device. In one aspect, the implant devices can be used to alter the pharmacokinetics of the bioactive agent.

Compositions comprising the implant devices can be administered to any desired subject. The subject can be a vertebrate, such as a mammal, a fish, a bird, a reptile, or an amphibian. The subject of the herein disclosed methods can be, for example, a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The compositions can also be administered by any suitable route, including parenterally, orally, among others. In one preferred aspect, the composition can be injected into subject.

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

What is claimed is:

1. An implant device comprising a biocompatible or biodegradable longitudinal body comprising a longitudinal surface and a proximal and distal end surface; wherein the implant device comprises a bioactive agent coated onto one or more surfaces and not dissolved or dispersed within the longitudinal body.

2. The implant device of claim 1, wherein the implant device comprises a bioactive agent coated only onto the proximal and/or distal end surface.

3. The implant device of claim 1, wherein the longitudinal body comprises poly(lactide), poly(glycolide), poly(caprolactone), poly(lactide-co-glycolide), or an admixture, combination, or copolymer thereof.

4. An implant device comprising a longitudinal body having an inner core comprising a longitudinal surface surrounded by a polymeric sheath and exposed proximal and distal end surfaces that are not surrounded by the polymeric sheath; wherein the polymeric sheath comprises a longitudinal outer surface which is substantially coextensive with the longitudinal core surface; and
wherein at least one of the inner core or the polymeric membrane sheath comprises a biodegradable polymer having a bioactive agent dissolved or dispersed therein.

5. The implant device of claim 4, wherein both the inner core and the polymeric membrane sheath comprise a bioactive agent dissolved or dispersed therein.

6. The implant device of claim 4, wherein both the inner core and the polymeric membrane sheath comprise a bioactive agent dissolved or dispersed therein; and wherein the inner core and the polymeric membrane sheath comprise different concentrations of bioactive agent.

7. The implant device of claim 4, wherein one or more of the proximal end surface, distal end surface, or longitudinal outer surface is coated with a biocompatible or biodegradable coating polymer.

8. The implant device of claim 4, wherein one or more of the proximal end surface, distal end surface, or longitudinal outer surface is coated with a bioactive agent that is the same or different than the bioactive agent dissolved or dispersed in the inner core and/or the polymeric membrane sheath.

9. The implant device of claim 4, wherein the polymeric membrane sheath does not comprise a bioactive agent dissolved or dispersed therein.

10. The implant device of claim 4, wherein the polymeric sheath comprises a polymer that creates a barrier membrane around the inner core.

11. The implant device of claim 4, wherein the longitudinal body comprises poly(lactide), poly(glycolide), poly(caprolactone), poly(lactide-co-glycolide), or an admixture, combination, or copolymer thereof.
